(19) United States
(12) Patent Application Publication
(10) Pub. No.: US 2010/0279402 A1
(43) Pub. Date: Nov. 4, 2010

(54) Expression Construct for Digesting Aggregating Protein and Method of Inhibiting the Aggregation of Aggregating Protein

(75) Inventors: Shin-ichi Yamada, Nagoya-shi (JP); Jyun-ichi Niwa, Aichi (JP); Gen Sobue, Aichi (JP)

Correspondence Address: EDWARDS ANGELL PALMER & DODGE LLP, P.O. BOX 55874, BOSTON, MA, 02205, US

(73) Assignee: National University Corporation Nagoya University, Nagoya (JP)

(21) Appl. No.: 12/225,311

(22) PCT Filed: Mar. 19, 2007
(86) PCT No.: PCT/JP2007/055493

§ 371 Date: Jul. 19, 2010

(30) Foreign Application Priority Data
Mar. 20, 2006 (JP) 2006-076789

(51) Int. Cl. C12N 5/071; C12N 15/79; C12N 5/07

(52) U.S. Cl. 435/366; 435/320.1; 435/325

(57) ABSTRACT

It is intended to provide a means which is efficacious in digesting a protein forming aggregates in a eukaryotic cell such as mutant superoxide dismutase 1 or an androgen receptor having an abnormally extended polyglutamine chain. Namely, an expression construct for digesting an aggregating protein which has a nucleic acid encoding an archaeal proteasome and being connected in an operable manner to a promoter for eukaryotic cells. By transferring this expression construct into a eukaryotic cell, the aggregating protein is digested owing to the action of the archaeal proteasome.

### TECHNICAL FIELD

The present invention relates to a novel use of an archaeal proteasome. More particularly, the present invention relates to an expression construct for degrading an aggregative protein using an archaeal proteasome, and a method of inhibiting aggregative proteins from forming aggregates.

A 20S proteasome is a “barrel shaped” proteolytic enzyme complex with high universality for degrading most of intracellular proteins (non-patent document 1) in which four rings formed of seven protein subunits are laminated (non-patent document 2). The α subunit forms an outer ring (non-patent document 3) and the β subunit having an ability of degrading protein forms an inner ring (non-patent document 5).

The ubiquitin—proteasome systems of eukaryotic cells degrade aberrant proteins that easily accumulate or proteins that are not folded well (non-patent document 6). It is thought that these intracellular abnormal aggregates are involved in the onset of neurodegenerative diseases such as Parkinson's disease, amyotrophic lateral sclerosis (ALS), polyglutamine disease (Huntington's disease, several types of spinocerebellor degeneration, sphere myelopathic muscular atrophy (SBMA)) and Alzheimer's disease, in which the hypofunction of proteasome is related to the pathologic condition (non-patent documents 7-11). However, the cause of the accumulation of abnormal proteins is not clear. It is thought that the solution of this common problem will lead to establishment of a great treatment method.

[Non-patent document 1] Hershko, A. Ciechanover, A. (1998) Annu. Rev. Biochem. 67, 425-479

[Non-patent document 2] Puhler, G., Weinkauf, S., Bachmann, L., Muller, S., Engel, A., Hegerl, R., Baumeister, W. (1992) EMBO J. 11, 1607-1616

[Non-patent document 3] Zwickl, P., Kleinz, J., Baumeister, W. (1994) Nature Struct. Biol. 1, 765-770

[Non-patent document 4] Seemuller, E., Lupas, A., Stock, D., Lowe, J., Huber, R., Baumeister, W. (1995) Science 268, 579-582

[Non-patent document 5] Grziwa, A., Baumeister, W., Dahlmann, B., Kopp, F. (1991) FEBS Lett. 290, 186-190

[Non-patent document 6] Ciechanover A, Orian A, Schwartz A L. (2000) J. Cell Biochem. 77, 40-51

[Non-patent document 7] Kabashi, E., Agar, J. N., Taylor, D. M., Minotti, S., Durham, H. D. (2004) J. Neurochem. 89, 1325-35

[Non-patent document 8] Bailey, C. K., Andriola, I. F., Kampinga, H. H. and Merry, D. E. (2002) Hum. Mol. Genet. 11, 515-523

[Non-patent document 9] Chen, Q., Thorpe, J., Keller, J. N., (2005) J. Biol. Chem. 26, 30009-30017

[Non-patent document 10] Keck, S., Nitsch, R., Grune, T., Ullrich, O. (2003) J. Neurochem. 85, 115-122

[Non-patent document 11] Bence, N. F., Sampat, R. M. and Kopito, R. R. (2001) Science 292, 1552-1555

[Non-patent document 12] Baumeister, W., Walz, J., Zuhl, F., Seemuller, E. (1998) Cell 92, 367-380

[Non-patent document 13] Zwickl, P., Goldberg, A. L., Baumeister, W. (2000) Proteasomes: The World of Regulatory Proteolysis, Landes Bioscience, Georgetown, Tex.

[Non-patent document 14] Zwickl, P., Ng, D., Woo, K. M., Klenk, H. P., Goldberg, A. L. (1999) J. Biol. Chem. 274, 26008-26014

[Non-patent document 15] Venkatraman, P., Wetzel, R., Tanaka, M., Nukina, N., Goldberg, A. L. (2004) Mol. Cell 14, 95-104

### DISCLOSURE OF THE INVENTION

### Problems to be Solved by the Invention

Proteasome α and β subunits of archaeal 20S have only one type, respectively, and they are thought to be a progenitor of the proteasome of eukaryotic cells (non-patent document 12). On the other hand, proteasome α and β subunits of eukaryotic cells are composed of seven different types of subunits (non-patent document 12). It is thought that the archaea does not have a ubiquitin recognition system acting on the degradation of protein and that there is the other unknown tag (non-patent document 13). Furthermore, it is thought that the archaea have a complex for preparing 20S proteasome called proteasome-activating nucleotidase (PAN) as a progenitor of 19S of eukaryotic cells. It is thought that the PAN forms a complex corresponding to the lower part of the 19S and is necessary for efficient protein degradation by the 20S (non-patent document 14). However, it is shown that the archaeal proteasome can rapidly degrade a polyglutamine aggregate without PAN in vitro (non-patent document 15).

Under such circumstances, an object of the present invention is to provide a means effective to degrade a protein that forms an aggregate in eukaryotic cells, for example, a mutant superoxide dismutase 1, an androgen receptor having an abnormally extended polyglutamine chain, and the like.

### Means for Solving Problems

In order to examine whether or not the degradation by an archaeal proteasome without depending upon PAN can be carried out in eukaryotic cells, the present inventors have carried out an experiment using a 20S proteasome of Methanosarcina mazei (Mm) growing at 37° C. suitable for experiments for culturing cells. As a result, by generating the archaeal 20S proteasome in eukaryotic cells in a state in which the archaeal 20S proteasome has a function, the present inventors have succeeded in degrading superoxide dysmutase-1 (SOD1) that is a causative protein of familial ALS or an androgen receptor (AR) having an extended polyglutamine chain that is a causative protein of SBMA in a mutant-specific way and in reducing the cytotoxicity thereof. Furthermore, it has been clarified that this archaeal proteasome can degrade proteins such as α-synuclein and tau, which are related to other neurodegenerative diseases. Thus, it has been determined that the archaeal proteasome is useful for degrading an aggregative protein in eukaryotic cells, thus opening a way to establishing new treatment for diseases caused by the toxicity of abnormal proteins accumulated in cells.

The present invention provides a below-mentioned expression construct for degrading aggregative protein and a method of inhibiting aggregative proteins forming aggregates and the like based on the above-mentioned findings and results.
[1] An expression construct for degrading an aggregative protein containing a nucleic acid sequence encoding a proteasome of archaea and being operatively connected to a promoter for a eukaryotic cell.
[2] The expression construct described in [1], wherein the nucleic acid sequence encodes archaeal proteasome α subunit and/or β subunit.
[3] The expression construct described in [2], wherein the α subunit includes an amino acid sequence set forth in SEQ ID NO: 1 or an amino acid sequence that is different from the amino acid sequence set forth in SEQ ID NO: 1 only in a part that does not substantially affect a function of the proteasome α subunit; and the β subunit includes an amino acid sequence set forth in SEQ ID NO: 3 or an amino acid sequence that is different from the amino acid sequence set forth in SEQ ID NO: 3 only in a part that does not substantially affect a function of the proteasome βsubunit.
[4] The expression construct described in [1], wherein the nucleic acid sequence includes a DNA sequence set forth in SEQ ID NO: 2 and/or a DNA sequence set forth in SEQ ID NO: 4.
[5] The expression construct described in [1] or [2], wherein the archaea belongs to the Methanosarcina genus.
[6] The expression construct described in [1] or [2], wherein the archaea is Methanosarcina mazei.
[7] The expression construct described in any of [1] to [6], wherein the promoter for a eukaryotic cell is a promoter for a mammalian.
[8] The expression construct described in any of [1] to [7], wherein the aggregative protein is a protein selected from the group consisting of mutant superoxide dismutase 1, an androgen receptor having an abnormally extended polyglutamine chain, α-synuclein and tau.
[9] A method of inhibiting an aggregative protein from forming an aggregate in a target eukaryotic cell, the method comprising introducing the expression construct described in any of [1] to [8] into the target eukaryotic cell.
[10] A method of inhibiting an aggregative protein from forming an aggregate in a target eukaryotic cell, the method comprising forcedly expressing archaeal proteasome α subunit and β subunit in the target eukaryotic cell.
[11] The method described in [10], wherein the archaea belongs to the Methanosarcina genus.
[12] The method described in [10], wherein the archaea is Methanosarcina mazei.
[13] The method described in any of [9] to [12], wherein the target eukaryotic cell is a human cell.
[14] The method described in any of [9] to [12], wherein the target eukaryotic cell is an isolated human cell.
[15] The method described in any of [9] to [12], wherein the target eukaryotic cell is a non-human mammalian cell.
[16] A use of a proteasome of archaea to prepare an expression construct for degrading an aggregative protein or to inhibit an aggregative protein from forming an aggregate in a target eukaryotic cell.

### BRIEF DESCRIPTION OF THE DRAWINGS

FIG. 1 shows expression of the Mm proteasome in a eukaryotic cell. (A) A configuration view of an expression vector used in this experiment, showing a deletion site in the Δα subunit. A Thr1Cysβ subunit (mβ1) is prepared by substituting three bases. (B) Western blotting analysis using an anti-α subunit antibody, an anti-β subunit antibody, and an anti-His-tag antibody. (C) Ni-NTA pull down analysis in which immunoprecipitation is carried out by the anti-α subunit antibody. (D) A chymotrypsin-like activity of a Ni-NTA pull down sample in which an error bar shows s.d. (n=3). (E) Glyceol concentration gradient ultracentrifugation showing that the Mm proteasome α and β subunits are located in the fractions that are substantially the same as those of the endogenous human 20S proteasome α1 and α5 subunits.

FIG. 2 shows that the expression amount of mutant SOD1 is reduced in the presence of Mm proteasome αβ. 1 μg of SOD1-MycHis vector and Mm proteasome subunit are transfected to Neuro2a that have been cultured in a 6 cm-culture dish, which is analyzed after 48 hours. It is shown that the expression amount of the mutant SOD1 is gradually reduced as the increase in the amount of the Mm proteasome αβ. The Mm proteasome αmβ1 does not show such an effect. WT: wild type SOD1, G93A: SOD1^{G93A}, G85R: SOD1^{G85R}, G37R: SOD1^{G37R}, H46R: SOD1^{H46R}.

FIG. 3A shows that Mm proteasome αβ promotes the degradation of the mutant SOD1, showing the result of the cycloheximide chase analysis (see, method). It is shown that the degradation of various mutants SOD1 is promoted in the presence of the Mm proteasome αβ. Graphs show continuous three times of data of SOD1^{G93A} and SOD1^{G85R}. An error bar shows s.d.

FIG. 3B shows the result of Pulse chase analysis (see, method). It is shown that the degradation of SOD1^{G93A} is promoted in the presence of the Mm proteasome αβ. Circle mark: mock, triangle mark: αβ, and square mark: αmβ1. An error bar shows s.d (n=3).

FIG. 4 shows that the Mm proteasome αβ reduces the cytotoxicity by mutant SOD1, showing a dose-dependent effect of the Mm proteasome αβ on the toxicity of SOD1. The HEK293 cytotoxicity by (A) wild type SOD1, (B) mutant SOD1^{G93A} and (C) mutant SOD1^{G85R} are analyzed by using 3-(4,5-dimethylthiazol-2-yl)-5-(3-carboxymethoxyphenyl)-2-(4-sulfophenyl)-2H-tetrazolium (MTS). A transverse line in the box shows the average; upper line and lower line of the box show 75 percentile and 25 percentile, respectively; and T-bars in the upper and lower parts show 90 percentile and 10 percentile, respectively (n=3×6 wells). The values show the amount of transfected DNA (for example, αβ 0.1=α 0.05 μg+β 0.05 μg). (D) shows the relative comparison of caspase 3/7 activities using a fluorescence substrate Z-DEVD-R110. The Mm proteasome αβ shows the activation of the caspase 3/7. Positive control shows 3.2±0.2 (cells incubated for 24 hours with 1 μM staurosporin are used).

FIG. 5 shows that the mutant SOD1 and the Mm proteasome αβ coexist in cells. GFP-tagged wild type SOD1 or mutant SOD1^{G93A} and the Mm proteasome αβ are transfected to HEK293 cells and fixed after 48 hours. An anti-His antibody is used as a primary antibody, and an Alexa-546 anti-mouse antibody is used as a secondary antibody. WT: wild type SOD1, G93A: SOD1^{G93A}.

FIG. 6 shows that the Mm proteasome αβ promotes the degradation of a mutant androgen receptor (AR) having an extended polyglutamine chain and reduces the cytotoxicity. (A) 1 μg of pCR3.1-AR24Q vector or pCR3.1-AR97Q vector and the Mm proteasome subunit are transfected to Neuro2a that has been cultured in a 6 cm-culture dish, which is analyzed after 48 hours. The expression amount of the mutant AR97Q is gradually reduced as the increase in the amount of the Mm proteasome αβ but AR24Q is not affected. The Mm proteasome αmβ1 does not show such an effect. (B) Results of cycloheximide chase analysis (see, method). The degradation of the mutant AR-97Q is promoted in the presence of the Mm proteasome αβ. (C) The Mm proteasome αβ reduces the cytotoxicity by AR-97Q. A transverse line in the box shows the average; upper line and lower line of the box show 75 percentile and 25 percentile, respectively; and T-bars in the upper and lower parts show 90 percentile and 10 percentile, respectively (n=3×6 wells).

FIG. 7 shows that the Mm proteasome αβ degrades a protein that forms an aggregate easily, but does not degrade a protein that does not easily form aggregates. The Mm proteasome subunit and (A) 1 μg of α-synuclein vector (wild type, A53T, and A30P), (B) tau vector (six isoforms: one in which a tubulin binding domain is repeated three times (3L, 3M, and 3S), one in which a tubulin binding domain is repeated four times (4L, 4M, and 4S), one in which two of 29 amino acids are included in the N terminal (3L and 4L), one in which one of them is included (3M and 4M), and one in which none of them are included (3S and 4S)), and (C) mock, GFP vector, and LacZ-V5 vector are transfected to Neuro2a that has been cultured in a 6-cm culture dish, which is analyzed. The expression amounts of (A) and (B), α-synuclein and tau, are reduced when they are expressed together with the Mm proteasome αβ. The expression amount of (C) endogenous GAPDH, exogenous GFP and LacZ are not affected by the expression of the Mm proteasome αβ.

### BEST MODE OF CARRYING OUT THE INVENTION

### (Terms)

For convenience, certain terms employed in the specification are collected herein.

Archaea, along with Bacteria and Eukarya, is one of three major divisions in the classification of living organisms. Archaea is characterized in that a component of the cell membrane is ether lipid; the position in which hydrocarbon is bonded to glycerol of a lipid skeleton is sn-2, 3 position; peptide glycan is not generally observed in the cell wall; it has a unique sensibility to antibiotics; and it has sensitivity to deiphteria toxin, and the like. Archaea includes Euryarchaeota phylum regnum), Crenarchaeota phylum (regnum), Korarchaeota phylum (regnum), and Nanoarchaeota phylum (regnum). Typically known examples of archaea include methane bacteria (Methanothermus fervidus, Methanococcus voltae, Methanobacterum formicicum, Methanococcus jannaschii, Methanosarucina mazei and the like), extremely halophilic bacteria (Halobacterium salinarum, and the like), thermophilic bacterium (Thermoplasma acidophilum), sulfur bacteria, and the like. As to the classification and identification method, see, for example, the following documents: (1) Archaea, A laboratory manual, edited by F. T. Robb, A. R. Place, K. R. Sowers, H. J. Schreier, S. DasSarma and E. M. Fleischmann, Cold Spring Harbor Laboratory Press, New York (1995), (2) Superbugs, Microorganisms in Extreme Environments edited by K. Horikoshi and W. D. Grant, Springer-Verlag, Tokyo (1991), (3) Extremophiles, Microbial Life in Extreme Environments edited by K. Horikoshi and W. D. Grant, Wiley-Liss, Inc., New York (1991).

In the specification, the terms “comprise/include” and “comprising/including” are used to include the meaning of “consisting of.” Therefore, for example, “a product (or method) comprising/including a plurality of elements (members)” necessarily includes also the terms “a product (or method) consisting of a plurality of elements (members)”

The term “disease” herein is used interchangeably with the terms meaning not normal conditions, for example, as illness, sickness, or pathologic condition, or the like.

As used herein, the term “nucleic acid” is intended to include DNA (including cDNA and genome DNA), RNA (including mRNA), DNA analog, and RNA analog unless otherwise noted. A nucleic acid in the present invention may have any forms, that is, it may be single-stranded or double-stranded, but preferably is double-stranded DNA. Furthermore, the degeneracy of codon is also considered. That is to say, in the case of a nucleic acid is nucleic acid encoding protein, the nucleic acid may have any base sequence as long as the protein can be obtained as the expression product.

The term “isolated nucleic acid” as used in this specification typically refers to nucleic acid in a state which is separated from the other nucleic acid coexisting in nature in a case of nucleic acid originally occurring in nature (for example, nucleic acid in a human living body). However, the nucleic acid may include a part of the other nucleic acid, for example, a part of the flanking nucleic acid sequence in the natural state. For example, in the case of genome DNA, the preferable embodiment of the “isolated nucleic acid” does not substantially include other DNA component, coexisting in the natural state (including an adjacent DNA sequence in the natural state) is not substantially contained.

For example, “isolated nucleic acid” such as a cDNA molecule, which is produced by a recombinant technology, is preferably a nucleic acid that is substantially free of other cellular components, culture solution, and the like. Similarly, when a nucleic acid is produced by chemical synthesis, “isolated nucleic acid” is preferably a nucleic acid in a state which is substantially free of chemical precursors (raw materials) or other chemical substances to be used in the synthesizing process.

Nucleic acid that is present as a part of a vector or a composition, or nucleic acid that is present in a cell as an exogenous molecule can be referred to as “isolated nucleic acid” as long as it is present as a result of artificial manipulation.

Unless otherwise noted, simply described “nucleic acid” in this specification means nucleic acid in an isolated state.

### (Expression Construct for Degrading Aggregative Protein)

The first aspect of the present invention relates to an expression construct for degrading an aggregative protein. In the present invention, “aggregative protein” is protein that exists singly in a normal state but obtains a property for forming an aggregate for some reasons or protein that is directed to form an aggregate and the aggregate exhibits cytotoxicity. Note here that the term “exists singly” herein means that an aggregate is not formed. A protein forming a complex together with the other molecule in a normal state corresponds to a protein that “exists singly” even if the protein forms such a complex. Furthermore, “cytotoxicity” refers to a negative property or effect with respect to the maintenance of the normal state of cells. A typical example of such a negative property or effect includes a property or effect for reducing the cell function or eliciting cell death.

An example of the aggregative protein can include mutant superoxide dismutase 1 (SOD1), an androgen receptor (AR) having an abnormally extended polyglutamine chain, α-synuclein, tau, amyloid-forming protein, prion protein, and the like. The mutant SOD1 is a causative protein of familial amyotrophic lateral sclerosis (familial ALS). Meanwhile, AR having an abnormally extended polyglutamine chain is a causative protein of sphere myelopathic muscular atrophy (SBMA). Furthermore, α-synuclein and tau are involved in the onset and development of Parkinson's disease and Alzheimer's disease, respectively. The abnormal accumulation of the α-synuclein and tau is observed in a patient's nerve cells. The expression construct of the present invention is typically used for the purpose of degrading a protein involved in such nerve cell diseases, and is useful for the treatment, prophylaxis and study of the critical mechanism, and the like.

The “expression construct” of the present invention contains a nucleic acid sequence encoding an archaeal proteasome (hereinafter, also referred to as “proteasome nucleic acid sequence”). In other words, the proteasome nucleic acid sequence contained in the expression construct of the present invention includes: (1) a nucleic acid sequence encoding an archaeal proteasome α subunit; (2) a nucleic acid sequence encoding an archaeal proteasome β subunit; or (3) a nucleic acid sequence encoding archaeal proteasome α and β subunits (in this case, an intervening sequence capable of expressing the proteasome β subunit, for example, IRES (internal ribosomal entry site) and the like, is disposed between a part encoding the proteasome α subunit and a part encoding the proteasome β subunit).

A preferable embodiment of the present invention uses the archaeal proteasome belonging to the Methanosarcina genus. Many of Archaea of Methanosarcina genus can grow in relatively mild temperature conditions, a part of which is used for producing methane. Methanosarcina mazei used in the below-mentioned Example can grow well in the temperature conditions of about 37° C. which is suitable condition for the existence of mammalian cells. Therefore, Methanosarcina mazei is preferable as an origin of the proteasome to be used in the present invention. That is to say, in a further preferable embodiment of the present invention, an expression construct incorporating a nucleic acid encoding proteasome α subunit and/or β subunit of the Methanosarcina mazei is constructed.

Note here that archaea are available from, for example, Independent Administrative Institution RIKEN (Rikagaku Kenkyūsho), Bio Resource Center, Independent Administrative Institution, National Institute of Technology and Evaluation, ATCC (American Type Culture Collection), DSMZ (German Collection of Microorganisms and Cell Cultures), and the like.

Specific examples of the proteasome nucleic acid sequence to be incorporated in the expression construct of the present invention can include: (1) amino acid sequence set force in SEQ ID NO: 1 (sequence of proteasome α subunit of the Methanosarcina mazei); and (2) amino acid sequence set force in SEQ ID NO: 3 (sequence of proteasome β subunit of the Methanosarcina mazei). Furthermore, the nucleic acid sequence of the above-mentioned (1) and the nucleic acid sequence of the nucleic acid sequence of (2) may be used together. In this case, it is possible to obtain an expression construct capable of forcedly expressing the proteasome α subunit and β subunit of Methanosarcina mazei in the target cells. Note here that the specific example of the nucleic acid sequence of the above mentioned (1) (DNA sequence) is shown in SEQ ID NO: 2 and the specific example of the nucleic acid sequence of the above mentioned (2) (DNA sequence) is shown in SEQ ID NO: 4, respectively.

The proteasome nucleic acid sequence can be prepared in an isolated state by using a standard genetic engineering technique, molecular-biological technique, biochemical technique, and the like, with reference to the sequence information disclosed in this specification or the attached sequence listing. For example, a proteasome nucleic acid sequence having a base sequence set force in SEQ ID NO: 2 (nucleic acid sequence encoding proteasome α subunit) can be amplified and isolated by carrying out nucleic acid amplification reaction (for example, PCR) using a hybridization method using the entire or part of the base sequence or the complementary sequence with a genome DNA of Methanosarcina mazei (ATCC BAA-159D) as a template. The proteasome nucleic acid sequence (a nucleic acid sequence encoding proteasome the β subunit) having a base sequence set forth in SEQ ID NO: 3 can be prepared in an isolated state by the similar method. In general, an oligonucleotide primer can be easily synthesized by using a commercially available automated DNA synthesizer.

In another embodiment of the present invention, an expression construct is constructed by using a nucleic acid sequence encoding a protein having the same function but partially different in the amino acid sequence (the protein is also referred to as “homologous α subunit”) when compared with a protein having the amino acid sequence set forth in SEQ ID NO: 1 (α subunit), and/or a nucleic acid sequence encoding a protein having the same function but partially different in the amino acid sequence (the protein is also referred to as “homologous β subunit”) when compared with a protein having the amino acid sequence set forth in SEQ ID NO: 3 (α subunit). Thus, in the present invention, an expression construct may be constructed by using a nucleic acid sequence encoding a protein that is regarded as being substantially the same in terms of the function as a protein having the amino acid sequence set forth in SEQ ID NO: 1 (in other words, as compared with the amino acid sequence, only a part that does not affect the function of the proteasome α subunit is different) and/or a nucleic acid sequence encoding a protein that is regarded as being substantially the same in terms of the function as a protein having the amino acid sequence set forth in SEQ ID NO: 3 (in other words, as compared with the amino acid sequence, only a part that does not affect the function of the proteasome β subunit is different). With such a nucleic acid construct, similar to the expression construct incorporating the nucleic acid sequence encoding the amino acid sequence set forth in SEQ ID NO: 1 and/or the nucleic acid sequence encoding the amino acid sequence set forth in SEQ ID NO: 3, a protein functioning as the proteasome α subunit in a target cell and/or a protein functioning as the proteasome β subunit can be forcedly expressed.

Herein, the term “amino acid sequence is different in part” as used herein denotes that the amino acid sequence is mutated (changed) by deletion, substitution of one to several amino acids constituting the amino acid sequence, or addition, insertion of one to several amino acids, or the combination thereof. The difference of the amino acid sequence herein is permitted as long as the function of the proteasome α subunit (when the amino acid set force in SEQ ID NO: 1 is modified) or the proteasome β subunit (when the amino acid set force in SEQ ID NO: 3 is modified) is maintained. As long as this condition is satisfied, the position in which the amino acid sequence is different is not particularly limited. Furthermore, the difference may occur in a plurality of positions. The “plurality of” as used herein denotes a number corresponding to less than about 30% of the entire amino acids, preferably a number corresponding to less than about 20%, yet further more preferably a number corresponding to less than about 10%, more preferably about less than about 5%, and most preferably less than about 1%. That is to say, the homologous α subunit has, for example, about 70% or more, preferably about 80% or more, further more preferably about 90% or more, yet further preferably about 95% or more, and most preferably about 99% or more of identity with respect to the amino acid sequence set forth in SEQ ID NO: 1. Similarly, the homologous β subunit has, for example, about 70% or more, preferably about 80% or more, further more preferably about 90% or more, yet further preferably about 95% or more, and most preferably about 99% or more of identity with respect to the amino acid sequence set forth in SEQ ID NO: 3.

Preferably, by allowing the conservative amino acid substitution to be generated in non-essential amino acid residues (amino acid residues that are not involved in the function of the α subunit or the β subunit), the homologous protein is obtained. Herein, “conservative amino acid substitution” refers to substitution of one amino acid residue with an amino acid residue having a similar side chain. The amino acid residue is classified into some families depending upon the side chain. The side chain includes a basic side chain (for example, lysine, arginine, and histidine), an acidic side chain (for example, aspartic acid, and glutamic acid), an uncharged polar side chain (for example, asparagine, glutamine, serine, threonine, tyrosine, and cysteine), a nonpolar side chain (for example, glycine, alanine, valine, leucine, isoleucine, proline, phenylalanine, methionine, and tryptophan), a β-branched side chain (for example, threonine, valine, and isoleucine), and an aromatic side chain (for example, tyrosine, phenylalanine, and tryptophan). Preferably, the conservative amino acid substitution is a substitution between amino acid residues in the same family.

On the other hand, it is reported that the archaeal proteasome β subunit is involved in the activation of threonine 1 region, glutamic acid 17 regions, lysine 33 regions, aspartic acid 105 regions and aspartic acid 166 regions. When the nucleic acid sequence encoding the homologous β subunit is prepared, it is preferable to avoid modifying these regions.

Herein, the identity (%) between two amino acid sequences can be determined by the following procedure. Firstly, two sequences are aligned for optimum comparison of the two sequences (for example, a gap may be introduced in the first sequence so as to obtain an optimum alignment with respect to the second sequence). When a molecule (amino acid residue) at the specific position in the first sequence and a molecule in the corresponding position in the second sequence are the same, the molecules in the positions are defined as being identical. The identity between two sequences is a function of the number of identical positions shared by the sequences (i.e., identity (%)=number of identical positions/total number of positions×100), taking into account the number of gaps, and the length of each gap, which need to be introduced for optimal alignment of the two sequences.

The comparison and determination of identity between two sequences can be carried out by using a mathematical algorithm. A specific example of mathematical algorithm that can be used for comparing sequences include an algorithm described in Karlin and Altschul (1990) Proc. Natl. Acad. Sci. USA 87:2264-68 and modified by Karlin and Altschul (1993) Proc. Natl. Acad. Sci. USA 90:5873-77 but the algorithm is not limited to this. Such an algorithm is incorporated in NBLAST and XBLAST programs (version 2.0) of Altschul et al. (1990) J. Mol. Biol. 215: 403-10. BLAST polypeptide searches may be carried out by, for example, the XBLAST program, score=50, wordlength=3 to obtain amino acid sequence homologous to a certain amino acid sequence. To obtain gapped alignments for comparison purposes, Gapped BLAST as described in Altschul et al., (1997) Amino Acids Research 25(17): 3389-3402 can be utilized. When utilizing BLAST and Gapped BLAST programs, the default parameters of the respective programs (e.g., XBLAST and NBLAST) can be used. In detail, see http://www.ncbi.nlm.nih.gov. Another example of mathematical algorithm that can be used for comparing sequences includes an algorithm of Meyers and Miller (Comput. Appl. Biosci. 4: 11-17 (1988)) which has been incorporated into the ALIGN program that can be used for, for example, GENESTREAM network server (IGH Montpellier, France) or ISREC server. When the ALIGN program is used for comparison of the amino acid sequences, for example, a PAM120 weight residue table can be used with a gap length penalty of 12 and a gap penalty of 4.

The identity between two amino acid sequences can be determined using the GAP program in the GCG software package, using a Blossom 62 matrix or PAM250 matrix and a gap weight of 12, 10, 8, 6, or 4, and a gap length weight of 2, 3, or 4.

A promoter for eukaryotic cells is incorporated in the expression construct of the present invention, and the proteasome nucleic acid sequence is operably linked to the promoter. In the expression construct having this configuration, the proteasome nucleic acid sequence can be forced to be expressed in eukaryotic cells by the action of the promoter for eukaryotic cells. The term “ . . . is operably linked to the promoter” herein has the same meaning as “disposed under control of the promoter.” In general, the nucleic acid sequence encoding the archaeal proteasome is connected to the 3′ terminal side of the promoter directly or via other sequence.

In eukaryotic cell promoters, preferably, a mammalian cell promoter is used. Examples of the mammalian cell promoter may include CMV-IE (a promoter derived from a cytomegalovirus early gene), SV40ori, retrovirus LTP, SRα, EF1α, β actin promoter, and the like. Mammalian tissue specific promoters such as an acetylcholine receptor promoter, an enolase promoter, an L7 promoter, a nestin promoter, an albumin promoter, an alpha-fetoprotein promoter, a keratin promoter, an insulin promoter, and the like, can be used.

The nucleic acid construct may include an enhancer sequence or a selectable marker sequence. The use of the enhancer sequence can improve the expression efficiency of the proteasome nucleic acid sequence. Furthermore, when the expression construct containing a selectable marker sequence is used, it is possible to confirm the presence (and the degree thereof) of the introduction of the expression construct by using the selectable marker.

Note here that the insertion of the promoter, a proteasome nucleic acid sequence, an enhancer sequence (if necessary), a selectable marker sequence (if necessary), and the like, can be carried out by standard recombinant DNA technologies (see, for example, Molecular Cloning, Third Edition, 1.84, Cold Spring Harbor Laboratory Press, New York. A well-known method using an restriction enzyme and DNA ligase).

The expression construct of the present invention is used for introducing the proteasome nucleic acid sequence into the target cell. The form of the expression construct is not particularly limited as long as it can be used for the above-mentioned purpose, but the preferable form is an expression vector. The “expression vector” herein denotes a nucleic acid molecule capable of introducing an inserted nucleic acid into the target cells (host cells) to thereby express it in the cells. The expression vector includes a virus vector and a non-virus vector. The gene introduction method using a virus vector uses a phenomenon in which virus infects cells, and thus high gene introduction efficiency can be obtained. As a virus vector, an adenovirus vector, an adeno-associated virus vector, a retrovirus vector, a lentivirus vector, a herpesvirus vector, a Sendai virus vector, and the like, have been developed. Among them, when the adeno-associated virus vector, retrovirus vector, and lentivirus vector is used, a foreign gene incorporated in the vector is incorporated into the host chromosome, and thus stable and long-term expression can be expected. Since the retrovirus vector needs the cell division for incorporating the virus genome into the host chromosome, it is not suitably introduced into a non-dividing cell. On the other hand, a lentivirus vector or an adeno-associated virus vector are incorporated into the foreign gene even in non-dividing cells after infection. Therefore, these vectors are effective in expressing a foreign gene in non-dividing cells such as nerve cells or liver cells stably and for a long time.

Each virus can be prepared in accordance with the above-mentioned method or by using a commercially available dedicated kit. For example, the adenovirus vector can be produced by a COS-TPC method, a full-length DNA introduction method, or the like. The COS-TPC method is a method for preparing a recombinant adenovirus by simultaneously transfecting a recombinant cosmid including the intended cDNA or an expression cassette and a parent virus DNA-terminal protein complex (DNA-TPC) into 293 cells and using homologous recombination occurring in the 293 cells (Miyake, S., Makimura, M., Kanegae, Y., Harada, S., Takamori, K., Tokuda, C., and Saito, I. (1996) Proc. Natl. Acad. Sci. USA, 93, 1320.). On the other hand, the full-length DNA introduction method is a method of producing a recombinant adenovirus by carrying out a restriction digestion of a recombinant cosmid into which the target gene has been inserted, followed by transfection into the 293 cells (M. Terashima, S. Kondo, Y. Kanegae, I. Saito (2003) Journal of Experimental Medicine, 21 (7) 931.). The COS-TPC method can be carried out by using Adenovirus Expression Vector Kit (Dual Version) (TAKARA BIO INC.) and Adenovirus genome DNA-TPC (TAKARA BIO INC.). Furthermore, the full-length DNA introduction method can be carried out by using Adenovirus Expression Vector Kit (Dual Version) (TAKARA BIO INC.).

On the other hand, the retrovirus vector can be produced by the following procedures. Firstly, virus genomes (gag, poi, env genes) other than the packaging signal sequence between LTRs (Long Terminal Repeat) existing at both ends of the virus genome are removed, and the target gene is inserted therein. The thus constructed virus DNA is introduced into a packaging cells structurally expressing gag, pol, env genes. Thus, only a vector RNA having the packaging signal sequence is incorporated into the particle and a retrovirus vector is produced.

As a vector obtained by applying or modifying an adenovector, a vector whose specificity is improved by modifying a fiber protein (specific infection vector) or a gutted vector (helper dependent vector) in which the expression efficiency of the target gene is expected to be improved, and the like, have been developed. The expression vector of the present invention may be constructed as such a virus vector.

As the non-virus vector, a liposome, a positively charged liposome (Feigner, P. L., Gadek, T. R., Holm, M. et al., Proc. Natl. Acad. Sci., 84:7413-7417, 1987), a HVJ (Hemagglutinating virus of Japan)—liposome (Dzau, V. J., Mann, M., Morishita, R. et al., Proc. Natl. Acad. Sci., 93:11421-11425, 1996, Kaneda, Y., Saeki, Y. & Morishita, R., Molecular Med. Today, 5:298-303, 1999), and the like, have been developed. The expression vector of the present invention may be constructed as such a non-virus vector.

### (Method of Inhibiting Formation of Aggregates)

The second aspect of the present invention relates to a method of inhibiting an aggregative protein from forming an aggregate in a target cell by using an archaeal proteasome (hereinafter, referred to as “an inhibiting method of the present invention”). Note here that in the present invention, the terms “inhibition” can be used interchangeably with the teen “suppression.”

In one embodiment of the present invention, the above-mentioned expression construct of the present invention is used. That is to say, a step of introducing the expression construct of the present invention into a target cell is carried out. When the expression construct to be used contains a nucleic acid sequence encoding only the archaeal proteasome α subunit, when the expression construct is introduced into the target cell, a nucleic acid sequence encoding the archaeal proteasome β subunit is also introduced into the target cell. Thus, in the target cells, the proteasome α and subunits β derived from the different expression constructs are expressed, and the archaeal proteasome is constructed. On the other hand, when the expression construct to be used contains a nucleic acid sequence encoding the archaeal proteasome α and subunits β, by introducing it into the target cell, subunits α and β are expressed in the target cells. Thus, the archaeal proteasome is constructed.

The “target cells” herein are eukaryotic cells. Specific examples of the target cells include human cells, non-human mammalian cells such as monkey, mouse, and rat cells (COS cells, CHO cells, and the like), bacterial cell such as Escherichia coli, yeast cells, inset cells, and the like. Preferable target cells are mammalian cells, and particularly preferable target cells are nervous system cells (nerve cells and glia cells).

The inhibition method of the present invention is applied to isolated target cells or target cells constituting an organism individual. The term “isolated” used herein means a state in which it is taken out from the original environment (for example, a state constituting a living body). Therefore, in general, isolated target cells exist in a culture chamber or a preservation chamber, and artificial operations of the isolated target cells in vitro can be carried out. Specifically, cells (including cell strains) separated from a living body and being in a state which they are cultured outside a living body can be isolated target cells. Note here that even if they are in a state in which they constitute an organization, cells can be isolated cells as long as they are in the above-mentioned sense.

The isolated target cells can be prepared by a living individual. On the other hand, cells obtained from Independent Administrative Institution RIKEN (Rikagaku Kenkyūsho), Bio Resource Center, Independent Administrative Institution, National Institute of Technology and Evaluation, ATCC (American Type Culture Collection), DSMZ (German Collection of Microorganisms and Cell Cultures), and the like, can be used as the isolated target cells.

The introduction of target cells into the expression construct can be carried out by a calcium phosphate coprecipitation method, lipofenbufen (Feigner, P. L. et al., Proc. Natl. Acad. Sci. U.S.A. 84,7413-7417(1984)), an HVJ liposome method, a DEAE dextran method, an electroporation (Potter, H. et al., Proc. Natl. Acad. Sci. U.S.A. 81, 7161-7165(1984)), microinjection (Graessmann, M. & Graessmann, A., Proc. Natl. Acad. Sci. U.S.A. 73,366-370(1976)), a gene gun method, an ultrasonic gene introduction method, and the like, by taking types of the target cells, forms of the expression construct, and the like, into consideration. When a virus vector is used as an expression construct, the vector is introduced into a target cell by an infection.

The inhibition method of the present invention can be used for inhibiting the reduction in function of target cells by certain aggregates or the cell death (in other words, conservation of function or recovery of function). Therefore, the inhibition method of the present invention is an effective means for preventing or treating (that is, for the purpose of medical application) for diseases whose onset or development of pathologic condition is caused by formation of aggregates of specific protein. Thus, the inhibition method of the present invention can be used for gene therapy (or part thereof) with respect to specific diseases. Note here that a typical example of “diseases whose onset or development of pathologic condition is caused by formation of aggregates of specific protein” may include neurodegenerative disease such as familial ALS, SBMA, Parkinson's disease, and Alzheimer's disease.

Herein, the gene therapy includes a treating method by directly applying an expression construct for introducing gene into a patient body (in vivo gene therapy), and a treating method of introducing a gene into the cells collected from a subject to be treated and then applying the cells to the patient (ex vivo therapy). The inhibiting method of the present invention can be applied to both therapies. The administration route of the expression construct in the gene therapy in vivo is not particularly limited and may include, for example, local vaccination, intravenous, intracutaneous, subcutaneous, intramuscular and intraperitoneal injection, and the like. These administration routes are not excessively employed but may be used in arbitrary combination of two or more of them (for example, intravenous injection is carried out at the same time or a predetermined time after the oral administration, and the like). Herein, the “subject to be treated” is not particularly limited and may include human and non-human mammalian (pet animal, domestic animal, and laboratory animals). Specific examples of the subjects include mouse, rat, guinea pig, hamster, monkey, pig, cattle, goat, sheep, dog, cat, chicken, quail, and the like.). The subject to be treated by the inhibition method of the present invention is suitably human.

### (Expression Construct of the Present Invention or Other Application of Inhibition Method)

The expression construct of the present invention or the inhibition method of the present invention can be also used for the purpose of examining the behavior when the archaeal proteasome is forcedly expressed in certain eukaryotic cells. Furthermore, for the purpose of producing a transgenic non-human mammalian, the expression construct of the present invention and the like can be used. For example, a fertilized oocyte or an embryonic stem cell into which a nucleic acid encoding archaeal proteasome has been introduced by the expression construct of the present invention or the inhibition method of the present invention, so that a transgenic non-human mammalian can be generated. The non-transgenic animal of the present invention is useful in that the influence or effect of the archaeal proteasome on the mammalian can be investigated in the level of an individual. A transgenic non-human mammalian can be created by microinjection of directly introducing a nucleic acid into the pronuclei of a fertilized oocyte, a method of using a retrovirus vector, and a method of using ES cells. Hereinafter, a method using a microinjection method is described as one example of a method for producing a transgenic non-human mammalian.

In the microinjection method, firstly, a fertilized egg is collected from the oviduct of female mouse that is confirmed to have been mated, and the fertilized egg is cultured. Thereafter, an expression construct is injected into the pronuclei of the fertilized oocyte. The fertilized egg that has finished an infusion operation is transplanted into the oviduct of a pseudopregnant mouse and the mouse that has undergone transplantation is fed for a predetermined time so as to obtain a baby mouse (F0). In order to confirm that the chromosome of the baby mouse includes a transgene appropriately, DNA is extracted from, for example, the tail of the baby mouse and is subjected to a PCR method using a primer specific to the transgene or a dot hybridization method using a probe. The species of the “transgenic non-human mammalian” in this specification is not particularly limited. However, it is preferably a rodent such as a mouse, a rat, and the like.

Matters that are not described in this specification (conditions, operation methods, and the like) may follow the usual method. See, for example, Molecular Cloning (Third Edition, Cold Spring Harbor Laboratory Press, New York), Current protocols in molecular biology(edited by Frederick M. Ausubel et al., 1987).

### Example

### 1. Experimental Materials and Method

(1) Production of Expression Vector: Methanosarcina mazei (Mm) Proteasome Subunits α, β, Δ (2-13) α, and Mutant β (Thr1Cys)

By a PCR method using a genome of Mm (ATCC BAA-159D) purchased from ATCC as a template, the proteasome subunit α (Gene Bank GeneID: 1480962, Gene Bank Accession No. NP_{—}634644 (amino acid sequence, SEQ ID NO: 1) and Gene Bank Accession No. NC_{—}003901 (base sequence, SEQ ID NO: 2)) is amplified by using primers αF: 5′-GCGGGTACCCCACCATGCAGATGGCACCACAGATG (SEQ ID NO: 5) and αR: 5′-CGCCTCGAGTTATTCTTTGTTCTCATTTCCTTTGTG (SEQ ID NO: 6). The Δ(2-13)α (Δα) subunit is amplified by using ΔαF; 5′-GCGGGTACCCCACCATGACGGTTTTCAGCCCTGACGG (SEQ ID NO: 7) and the above-mentioned αR. The PCR product is inserted into the KpnI and XhoI sites of a pcDNA3.1 (+) vector (Invitrogen). The subunit β (Gene Bank GeneID: 1479036, Gene Bank Accession No. NP 632718 (amino acid sequence, SEQ ID NO: 3) and Gene Bank Accession No. NC_{—}003901 (base sequence, SEQ ID NO: 4)) is amplified by using βF: 5′-GCCTCTAGACCACCATGGATAATGACAAATATTTAAAG (SEQ ID NO: 8) and ΔR: 5′-GCGACCGGTGTTTCCTAAAGCTCTTCTG (SEQ ID NO: 9), and inserted into the XbaI and AgeI sites of the pcDNA3.1 (+) MycHis vector (Invitrogen) so that 6×histidine is connected to the C terminal. The mutant β subunit: mβ1 (Thr1Cys) is produced by using Site-directed Mutagenesis Kit (Stratagene) according to the attached manual. The pcDNA3.1/MycHis-SOD1 and pCMV-Tag4-SOD1 vector (wild type, G93A, G85R, H46R, and G37R) (reference document 16) and pEGFP-N1-SOD1 (wild type and G93A) vector, pCR3.1-AR24Q and pCR3.1-AR97Q vector, pcDNA3.1(+)/MycHis-α-synuclein (wild type, A53T and A30P)) are used from one that have been produced before (reference documents 16, 17 and 18). For six isoforms of tau proteins, the product, which has been amplified from a pRK172 vector provided from Dr. Michel Goedert, by the PCR method and then inserted into the KpnI and XhoI sites of the pcDNA 3.1(+) vector (Invitrogen), is used.

### (2) Cell Culture, Transfection, and Antibody

Neuro2a cells and Human embryonic kidney 293 (HEK293) cells are cultured by using Dulbecco's modified Eagle's medium (DMEM) containing 10% fetal calf serum. For the transfection in the MTS assay, Lipofectamine 2000 (Invitrogen) is used. For the transfection in the other experiments, Effectene Transfection Reagent (Qiagen) is used. Antibodies to be used listed below.

Anti-SOD1 antibody (SOD100, Stressgen bioreagents), anti-His antibody (Pentα-His, Qiagen), anti-α-tubulin antibody (clone B-5-1-1, Sigma), anti-20S proteasome β subunit antibody (derived from Methanosarcina thermophila, Calbiochem), anti-20S proteasome a subunit antibody (derived from Methanosarcina thermophila, Calbiochem), anti-AR antibody (N-20, Santa Cruz Biotechnology), anti-α-synuclein antibody (LB509, Zymed), and anti-tau antibody (Mouse Tau-1, Chemicon International).

### (3) Glyceol Concentration Gradient Ultracentrifugation

Cells cultured in a 10-cm diameter culture dish are recovered by using 1 ml of 0.01M Tris-EDTA (pH 7.5), subjected to thawing-melting cycle twice so as to destroy the cells, centrifuged at 4° C. for 15 minutes at 15000 g. The supernatant is infused into the top of 36 ml of glyceol having a 10-40% linear concentration gradient and centrifuged at 80000 g for 22 hours by using Beckman SW28 rotor. After centrifugation, 1 ml each is separated from the top into 37 fractions by using Liquid layer injector fractionator (LLIF) (Advantech, model number CHD255AA). 200 μl each of each fraction is precipitated in acetone. The precipitates are dissolved in 50 μl of sample buffer and used for SDS-PAGE and Western blotting. Immuno-stained bands are subjected to a qualification analysis by using ImageGauge software (FUJIFILM).

### (4) Ni-NTA Pull Down Assay

Mm proteasome subunits α, αβ, Δαβ and αmβ1 are transfected to HEK293 cells that have been cultured in a 10-cm diameter culture dish, and recovered in 1 ml of PBS buffer. The cells are subjected to thawing-melting cycle twice so as to destroy them, and centrifuged 3000 g. The supernatant is recovered. The supernatant and 200 μl of Ni-NTA agarose are mixed with each other and washed with 4 ml of 10 mM imidazole/PBS buffer four times, and thereafter, eluted with 2 ml of 250 mM imidazole/PBS buffer.

### (5) Measurement of Proteasome Activity

10 mM LLVY-AMC (Sigma) is added to 500 μl of sample produced by a Ni-NTA method and incubated at 37° C. for 12 hours, and the chymotrypsin-like activity is measured by using a multi-plate reader (PowerscanHT, Dainippon Seiyaku). The measurement is carried out three times and the analysis is carried out by using one-way ANOVA.

### (6) Immunocytochemistry

To HEK293 cells that have been cultured on a glass cover slip, pEGFP-N1-SOD1 and Mm proteasome α and β subunits are transfected. After 48 hours, the cells are fixed to be blocked and then incubated with the anti-His antibody at 4° C. over night. After washing, they are reacted with the secondary antibody (Alexα-546-anti-mouse antibody, Molecular Probes, Inc.) and photographed by using OLYMPUS BX51.

### (7) Cycloheximide Chase Analysis

One micron gram of pcDNA3.1/MycHis-SOD1 and mock (0.6 μg) or Mm proteasome αmβ1 (0.3 μg each), Mm proteasome αβ (0.3 μg each) are transfected to Neuro2a cells that have been cultured in a 6-cm diameter culture dish. After 24 hours, cycloheximide is added so as to become 50 μg/ml, cells are recovered at a predetermined time and used for SDS-PAGE and Western blotting.

### (8) Pulse Chase Analysis

One micron gram of pCMV-Tag4-SOD1^{G93A} and mock (0.6 μg) or Mm proteasome αmβ1 (0.3 μg each), Mm proteasome (0.3 μg each) are transfected to Neuro2a cells cultured in a 6-cm diameter culture dish. After 24 hours, it is labeled with (³⁵S) Cys for 60 minutes and recovered at a predetermined time. After immunoprecipitation by an anti-FLAG antibody (M2, Sigma), SDS-PAGE is carried out and the radioactivity is measured by Typhoon 9410 (Genaral Electric Company).

### (9) Cell Viability Analysis

HEK293 cells are cultured in a collagen-coated 96 well plate, pcDNA3.1/MycHis-SOD1 (wild type, G93A and G85r) and mock or Mm proteasome αmβ1 and Mm proteasome αβ (12 well each) are transfected. The cell viability analysis by 3-(4,5-dimethylthiazol-2-yl)-5-(3-carboxymethoxyphenyl)-2-(4-sulfophenyl)-2H-tetrazolium (MTS) is carried out 48 hours after the transfection. The absorbance in 490 nm is measured by using a multi-plate reader (PowerscanHT, Dainippon Seiyaku) with the temperature maintained at 37° C. The measurement is carried out three times and the analysis is carried out by using one-way ANOVA.

### (10) Caspase 3/7 Analysis

HEK293 cells are cultured in a 96 well plate, pcDNA3.1/MycHis-SOD1 (wild type, G93A and G85r), mock or Mm proteasome αmβ1, and Mm proteasome αβ are transfected, which is exchanged with a serum-free culture solution after 24 hours. After a further 24 hours, the analysis is carried out by using Apo-ONE Homogeneous Caspase-3/7 Assay (Promega) according to the attached manual.

### 2. Result

### (1) Cloning and Expression of Mm Proteasome

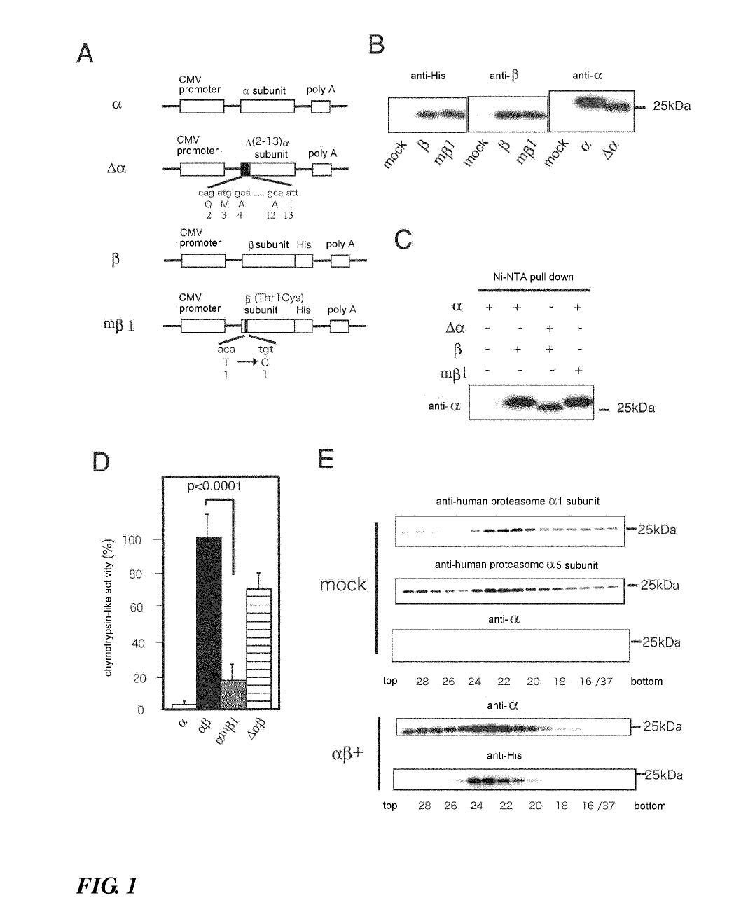

Mm proteasome α (Gene Bank GeneID: 1480962) and β (Gene Bank GeneID: 1479036) subunits are cloned from Mm genome, and furthermore, as shown in FIG. 1A, a Δ(2-13)α subunit (Δα) vector in which N-terminal 2-13 amino acids have been deleted is produced. The 2-13 amino acids are 20S proteasome and known to function as a gate for adjusting the input/output of the substrate (reference document 19). On the other hand, a mutant β subunit (Thr1Cys) is also produced. Thr1 of the archaeal proteasome β subunit is an activation center of the proteasome (reference document 20). The following experiments are carried out by using Neuro2a and HEK293 cells, and substantially the same results are obtained.

Firstly, in order to confirm the expression of the Mm proteasome subunit, mock, α, Δα, β, and mβ1 subunits are transfected to HEK293 cells and analyzed by Western blotting. FIG. 1B shows that the anti-α subunit antibody and the anti-β subunit antibody recognize the Mm proteasome α, Δα, and β subunits, respectively. It is shown that the endogenous proteasome subunit is recognized extremely slightly. The result of the Ni-NTA pull down analysis shows that α and Δα subunits are co-precipitated together with β or mβ1 subunit (FIG. 1C). Furthermore, as a result of the measurement of the proteasome activity of the precipitated samples, a chymotrypsin-like activity is significantly increased in the Mm proteasome αβ (FIG. 1D).

According to the glyceol concentration gradient ultracentrifugation, the Mm proteasome αβ, Δαβ, and αmβ1 subunits are classified in the fractions (20-25/37) the same as (human) proteasome α1 and α5 subunits and are estimated to have the molecular weight equivalent to that of human 20S proteasome. Furthermore, the band concentration detected in His antibody of 20-25 fractions occupies 80-90% of the total band concentration. The above-mentioned result shows that Mm proteasome α, Δα, β, and mβ1 subunits collectively formed a functional complex adequately. Since Mm proteasome Δαβ shows the toxicity, the following experiment is carried out by using Mm proteasome subunits αβ and αmβ1.

### (2) Mm Proteasome Degrades SOD1 in Mutant Specific Way

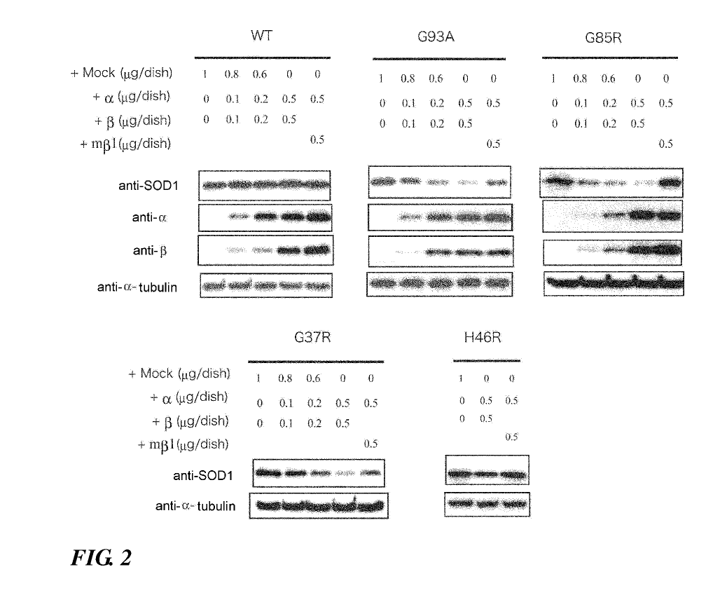

Next, we examined the effect of co-expression of Mm proteasome and mutant SOD1 proteins (SOD1^{G85R}, SOD1^{G37R}, SOD1^{G93A} and SOD1^{H46R}). In the cultured cell system, SOD1^{G8512}, SOD1^{G37R}, SOD1^{G93A} have stronger toxicity than SOD1^{H46R}. Furthermore, also in the familial ALS case, SOD1^{G85R}, SOD1^{G37R} and SOD 1^{G93A} show stronger condition than that of SOD1^{H46R}. Western blotting analysis shows that the expression amount of mutant SOD1 is reduced as increase in the expression amount of Mm proteasome αβ (FIG. 2). However, the wild type SOD1 is not affected by the expression of Mm proteasome αβ, and furthermore, the expression of the mutant SOD1 is not affected by the expression of the Mm proteasome αmβ1. This shows that the Mm proteasome activity is important in the change of the expression amount of the mutant SOD1. The reason why the degree of reduction in the expression amount of SOD1^{H46R} is small may be because the SOD1^{H46R} has smaller toxicity than the other mutants SOD1.

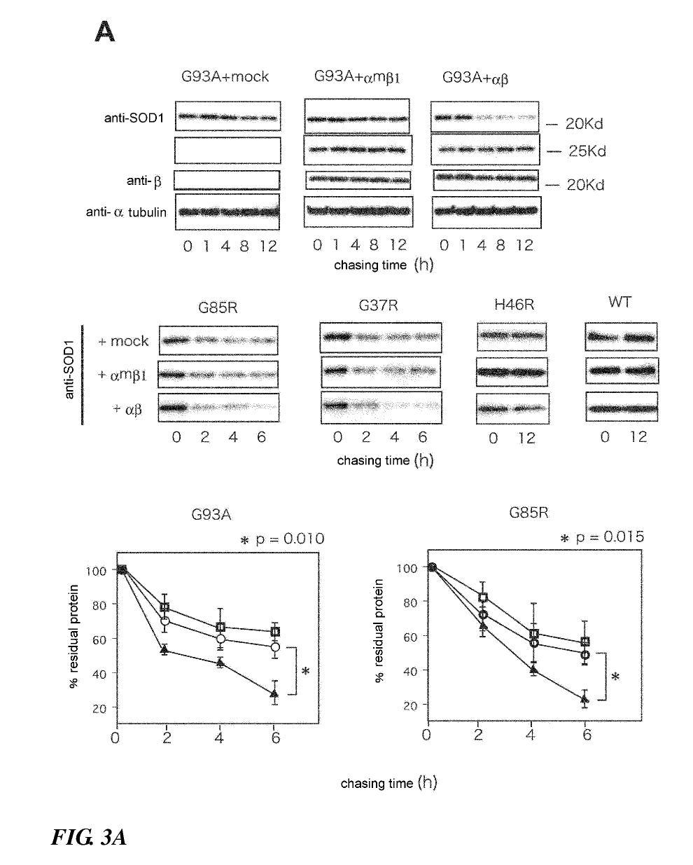

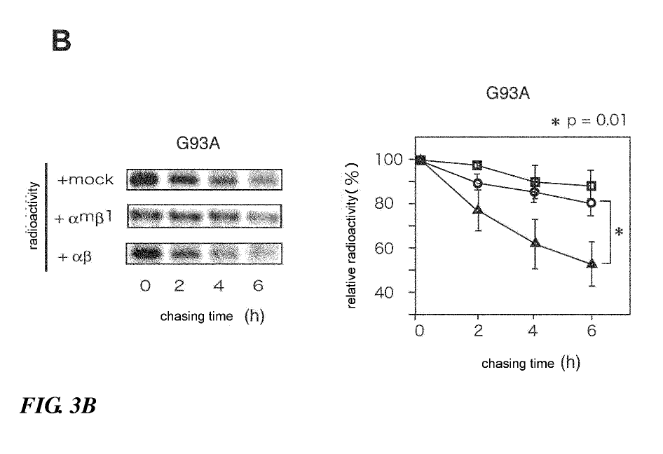

In order to examine whether the reduction in the expression amount of the mutant SOD1 is caused by the promotion of degradation or the reduction in the production amount, we investigated the degradation speed of the mutant SOD1 protein by using Neuro2a cells (FIGS. 3A and 3B). The chase analysis by using cycloheximide to stop the synthesis of protein shows that the degradation of SOD1 protein in a mutant dependent way (FIG. 3A). Furthermore, the change in the expression amount of the α and β subunits is not observed (FIG. 3A). The degradation of the wild type SOD1 is not affected by the expression of Mm proteasome αβsubunit. A pulse-chase analysis shows that the degradation of ³⁵S-labeled SOD1^{G93A} is significantly promoted in the cells expressed together with the Mm proteasome αβ subunit (FIG. 3B). These facts suggest that the activity center of the Mm proteasome has an extremely important function on the degradation of the mutant SOD1.

### (3) Mm Proteasome Reduces Cytotoxicity by Mutant SOD1

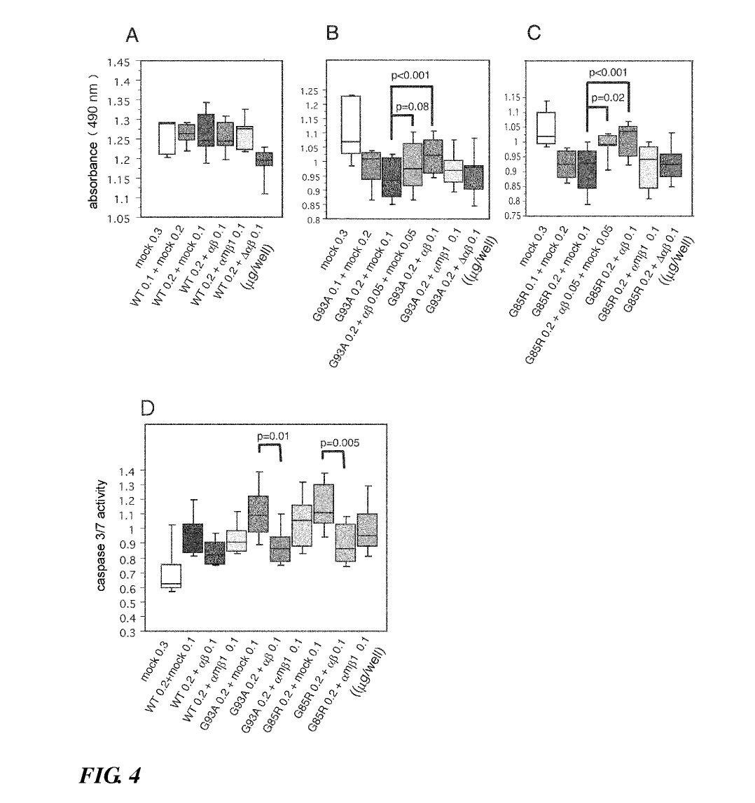

Next, we examined the change in the cytotoxicity when the Mm proteasome αβ, αmβ1 and mock and SOD1 (wild type, SOD1G85R, and SOD1G93A) are co-expressed in HEK293 cells by MTS analysis (FIG. 4). Even if the expression amount of the wild type SOD1 and Mm proteasome αβ are increased, the change in the cytotoxicity is not observed (FIG. 4). However, in the cells in which mutant SOD1 and Mm proteasome αβ are expressed, the cytotoxicity is increased in the expression dependent way of mutant SOD1, and the cytotoxicity is reduced in the expression amount dependent way of the Mm proteasome αβ (FIGS. 4B and 4C). Furthermore, this reducing effect is not confirmed in Mm proteasome αmβ1. The cytotoxicity of the mutant SOD1 is involved in the activation of caspase family, in particular, caspase 3 (reference document 21). The activity of caspase 3/7 when Mm proteasome αβ, αmβ1 and mock and SOD1 (wild type, SOD1^{G85R}, and SOD1^{G93A}) are co-expressed is examined by using the fluorescence substrate of caspase 3/7. It is shown that the Mm proteasome inhibits the activity of caspase 3/7 (FIG. 4D). The above-mentioned results show that the Mm proteasome αβ3 has an effect of reducing the cytotoxicity by mutant SOD1.

(4) Mm Proteasome Coexists with Intercellular Aggregate Formed by Mutant SOD1

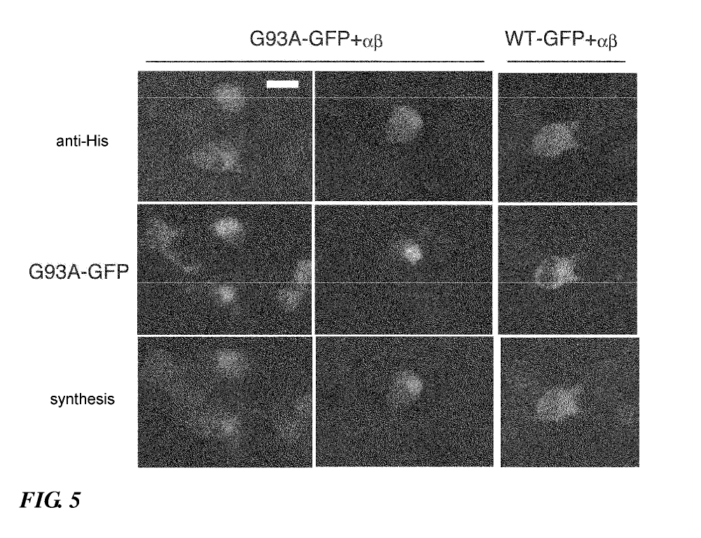

In the process of formation of an archaeal proteasome complex, the formation of a α ring is necessary to assembly of a β subunit (reference document 20). Furthermore, from the experimental results shown in FIG. 1E, since it is determined that most of the β subunits are used for forming a proteasome complex, the localization of the β subunits substantially corresponds to the localization of Mm proteasomes. Then, we examined the localization state of the Mm proteasome by using a His antibody. A GFP-tagged wild type SOD1 and a mutant SOD1^{G93A} vector are expressed together with the Mm proteasome αβ and fixed, followed by staining with anti-His antibody. As a result, GFP positive SOD1^{G93A} aggregates are positive to the His antibody. Furthermore, it is shown that the wild type SOD1 exists uniformly in the anti-His antibody and the cytoplasm (FIG. 5). Since GFP negative and anti-His antibody positive aggregates are not present, it is suggested that aggregates are formed together with the mutant SOD1. The similar results are observed in the Neuro2a cells.

### (5) Mm Proteasome Promotes Degradation of Androgen Receptor (AR) Having an Abnormally Extended Polyglutamine Chain and Reduces its Cytotoxicity

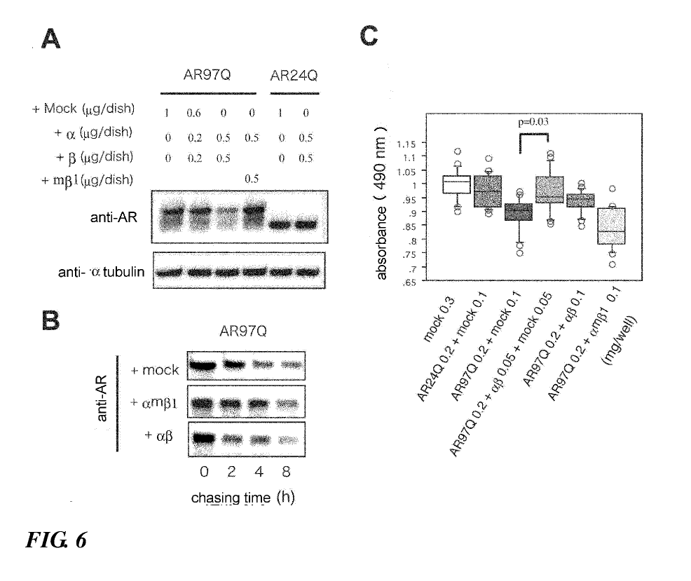

In order to examine whether the Mm proteasome can degrade a protein that forms an aggregate easily, we investigated an androgen receptor (97Q-AR) having 97 times repetition abnormally extended polyglutamine chains, which is a causative protein of SBMA. Similar to the results obtained in the SOD1 protein, the expression amount of 97Q-AR is reduced as the expression amount of Mm proteasome αβ is increased. However, even if the Mm proteasome αmβ1 is expressed, the expression of 97Q-AR is not affected (FIG. 6A). Furthermore, wild type AR (AR having 24 times repetition polyglutamine chains) does not show the change in the expression amount even if the expression amount of Mm proteasome αβ is increased. According to the cycloheximide chase analysis, the degradation of 97Q-AR is promoted in the presence of the Mm proteasome αβ. However, the change in the expression amount of 97Q-QAR in the presence of the Mm proteasome αmβ1 is not observed (FIG. 6B). In the MTS analysis, unlike the 24Q-AR, more toxicity is observed in 97Q-AR. However, the toxicity is reduced by the expression of the Mm proteasome αβ(FIG. 6C). These results show that the Mm proteasome αβ promotes the intercellular degradation of AR having an abnormally extended polyglutamine chain that easily forms an aggregate and reduces its cytotoxicity.

(6) Mm Proteasome αβ Promotes Degradation of Protein that Forms an Aggregate Easily, But Does Not Degrade a Protein that Does Not Easily Form an Aggregate.

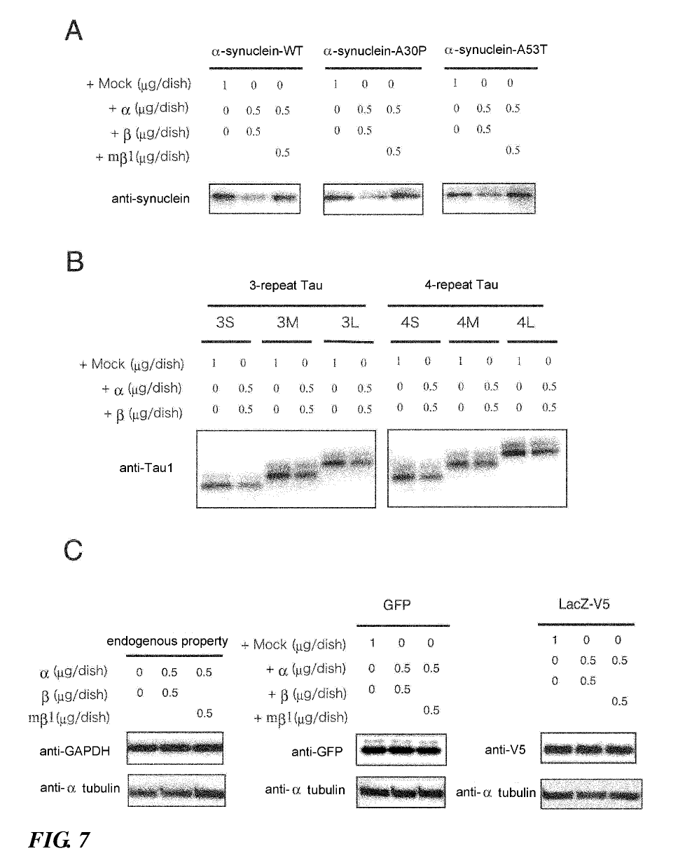

In order to investigate whether or not the degradation of the protein that easily forms an aggregate is promoted, an analysis is carried out by using α-synuclein (wild type, A53T, and A30P) and six isoforms of tau (which is divided into two types depending upon which the repetition number of the microtuble binding domains is three or four, and furthermore, and is divided into three types depending upon which the number of insertion of 29 amino acids at the N terminal is 2, 1, or 0. Six types in total). Similar to the results of the mutant SOD1 and 97Q-AR, in the presence of the Mm proteasome αβ, the expression amount of wild type, A53T, A30P, and all types of α-synuclein are reduced and all isoforms of tau are also reduced (FIGS. 7A and 7B). The expression of the wild type SOD1 and AR is not reduced even in the presence of the Mm proteasome αβ. However, α-synuclein and tau are reduced also in the wild type.

Next, we examined the change in the expression amount by allowing LacZ and GFP, which are difficult to form an aggregate, to form singly. However, the expression amount of these proteins is not changed even in the presence of the Mm proteasome αβ (FIG. 7C).

### 3. Consideration

In the present invention, we have shown that Mm proteasome α and β subunits of archaea form a functional proteasome complex having an ability to degrade protein and promote the degradation of protein, which forms an aggregate easily and is involved in the neurodegenerative diseases, in eukaryotic cells. The archaeal proteasome is originally assumed to have a chymotrypsin-like activity. However, the later researches have shown that the archaeal proteasome has various degradation activities and has 14 (7×2) activity centers (reference document 21). Furthermore, an archaeal proteasome is composed of one kind each of α and β subunits (reference document 6). According to the experiment for comparing the function of archaeal proteasome and the function of proteasome of eukaryotic cells in vitro, it is shown that the archaeal proteasome has much higher ability to degrade the polyglutamine chain than that of the proteasome of eukaryotic cells (reference document 9). We have tried to degrade protein that was not able to be degraded by the proteasome system of eukaryotic cells by the use of the potential ability and the ease in handling of the archaeal proteasome. The above-mentioned experimental results are the first to report that the archaeal proteasome promotes the degradation of the protein that easily forms an aggregate in eukaryotic cell.

Mm proteasome αβ promoted the degradation of mutant SOD1, 97Q-AR, wild type and mutant α-synucleins, and six isoforms of tau. In the former two (mutant SOD1 and 97Q-AR), the toxicity is shown in the cultured cell system, aggregates are formed in nerve cells causing the nerve cells to drop off so as to reduce the motor function in a transgenic mouse in which the proteins are excessively expressed. Therefore, they have been used as disease models of familial ALS and SBMA (reference documents 22 and 23). As a result of cycloheximide pulse chase analysis, it is shown that the Mm proteasome αβ promotes the degradation of SOD1 and AR in a mutant-dependent way.

However, α-synuclein and tau, even if they are a wild type, reduces the expression amount by the Mm proteasome αβ (FIG. 7). Herein, unlike SOD1 or AR, α-synuclein and tau are important that wild type protein is accumulated in Parkinson's disease and Alzheimer's disease, respectively. The aggregate of α-synuclein as Presynaptic protein are observed in sporadic and familial Parkinson's diseases such as synucleopaty, diffused Lewy body disease, multiple atrophy, and the like (reference document 24). In patient with sporadic Parkinson's disease, it is shown that wild type α-synucleins are accumulated and furthermore the expression amount is increased (reference document 25). The reduction in the function of proteasome prevents the degradation of α-synuclein, resulting in abnormally aggregating α-synuclein (reference document 26). Tau protein is observed as a neurufibrillary tangle in the neuron in Alzheimer's disease (reference document 27). The reduction in the function proteasome is reported in the brain of a patient with Alzheimer's disease (reference document 28). Both α-synuclein and tau protein have an aberrant three-dimensional structure relatively easily, which consequently may be led to aggregate (reference documents 29 and 30). Probably, it is thought that Mm proteasome αβ promotes the degradation of these proteins. Mm proteasome αβ is expected to promote the degradation of a wide range of proteins that easily form aggregates. On the contrary, the Mm proteasome αβ did not promote the degradation of GAPDH that are abundantly present or foreign GFP or LacZ that is relatively difficult to form aggregates.

The problem herein is that why such a degradation of protein that forms aggregates easily in a mutant-specific way and what recognition system is present. Archaeal 20S proteasome is said to be able to degrade the substrate efficiently by the action of PAN (reference document 8). The PAN is thought to be a progenitor of the bottom part of the eukaryotic cell 19S (reference document 8) and has a molecule chaperon-like function and a effect of unfolding an abnormally folded protein (reference document 31). A degradation recognition tag in archaea (like a ubiquitin tag in eukaryotic cells) has not been clarified. However, in vitro, archaeal 20S proteasome can degrade the polyglutamine chain very rapidly without the help of PAN (reference document 9). Herein, we have shown that the Mm proteasome αβ can degrade a protein that forms an aggregate easily without the help of PAN also in eukaryotic cells. The inner diameter of the proteasome is much smaller than that of abnormal protein aggregate (reference document 32). One hypothesis of the question of how an aggregate can enter the Mm proteasome αβis that the α ring itself works like a chaperon and it can recognize the aggregate and also unfold the aggregate. The gate of the α ring adjusts the entry of the substrate. There is an argument whether the gate in a natural state is open (reference document 33) or closed (reference documents 2 and 32). In our experiment, Mm proteasome Δαβ in which a gate of the α ring is deleted exhibits the cytotoxicity but the Mm proteasome αβ does not exhibit the toxicity. It is thought that the substrate is further efficiently degraded. This is impossible when the gate of the α ring is always closed. It is assumed that the Mm proteasome αβ approaches an aggregate, it unfolds the aggregate and further the ring is opened.

It has been reported that the molecule chaperons of Hsp 90, 70, 27, and the like, are involved in the degradation of mutant SOD1 or AR (reference documents 34 and 17). However, in our experiment at this time, the expression amount of the molecule chaperon is not changed, and the change of the ubiquitinated amount of SOD1 and AR, and the like has not been observed (data are not shown). It is not suggested that the results at this time have been obtained with the mechanism involved in degradation of such an endogenous protein.

With this experiment, we have shown in vivo that Mm proteasome αβ promotes the degradation of a protein that forms an aggregate easily involved in the neurodegenerative disease in a protein specific way. This characteristic effect is expected to be widely applied to diseases involved in aggregates.

### REFERENCE DOCUMENTS

1. Hershko, A. Ciechanover, A. (1998) Annu. Rev. Biochem. 67, 425-479
2. Puhler, G., Weinkauf, S., Bachmann, L., Muller, S., Engel, A., Hegerl, R., Baumeister, W. (1992) EMBO J. 11, 1607-1616
3. Zwickl, P., Kleinz, J., Baumeister, W. (1994) Nature Struct. Biol. 1, 765-770
4. Seemuller, E., Lupas, A., Stock, D., Lowe, J., Huber, R., Baumeister, W. (1995) Science 268, 579-582
5. Grziwa, A., Baumeister, W., Dahlmann, B., Kopp, F. (1991) FEBS Lett. 290, 186-190
6. Baumeister, W., Walz, J., Zuhl, F., Seemuller, E. (1998) Cell 92, 367-380
7. Zwickl, P., Goldberg, A. L., Baumeister, W. (2000) Proteasomes: The World of Regulatory Proteolysis, Landes Bioscience, Georgetown, Tex.
8. Zwickl, P., Ng, D., Woo, K. M., Klenk, H. P., Goldberg, A. L. (1999) J. Biol. Chem. 274, 26008-26014
9. Venkatraman, P., Wetzel, R., Tanaka, M., Nukina, N., Goldberg, A. L. (2004) Mol. Cell 14, 95-104
10. Ciechanover A, Orian A, Schwartz A L. (2000) J. Cell Biochem. 77, 40-51
11. Kabashi, E., Agar, J. N., Taylor, D. M., Minotti, S., Durham, H. D. (2004) J. Neurochem. 89, 1325-35
12. Bailey, C. K., Andriola, I. F., Kampinga, H. H. and Merry, D. E. (2002) Hum. Mol. Genet. 11, 515-523
13. Chen, Q., Thorpe, J., Keller, J. N., (2005) J. Biol. Chem. 26, 30009-30017
14. Keck, S., Nitsch, R., Grune, T., Ullrich, O. (2003) J. Neurochem. 85, 115-122
15. Bence, N. F., Sampat, R. M. and Kopito, R. R. (2001) Science 292, 1552-1555
16. Niwa, J., Ishigaki, S., Hishikawa, N., Yamamoto, M., Doyu, M., Murata, S., Tanaka, K., Taniguchi, N., Sobue, G. (2002) J. Biol. Chem. 277, 36793-36798
17. Waza, M., Adachi, H., Katsuno, M., Minamiyama, M., Sang, C., Tanaka, F., Inukai, A., Doyu, M., Sobue, G. (2005) Nature Med. 11, 1088-1095
18. Ito, T., Niwa, J., Hishikawa, N., Ishigaki, S., Doyu, M., Sobue, G. (2003) J. Biol. Chem. 278, 29106-29114
19. Benaroudj, N., Zwick, P., Seemuller, E., Baumeister, W., Goldberg, A. L. (2003) Mol. Cell 11, 69-78
20. Seemuller, E., Lupas, A., Baumeister, W. (1996) Nature 382, 468-471.
21. Dahlmann, B., Kopp, F., Kuehn, L., Hegerl, R., Pfeifer, G., Baumeister, W. (1991) Biomed. Biochim. Acta 50, 465-469
22. Gurney, M. E., Pu, H., Chiu, A. Y., Dal Canto, M. C., Polchow, C. Y., Alexander, D. D., Caliendo, J., Hentati, A., Kwon, Y. W., Deng, H. X., Chen, W., Zhai, F., Sufit, R. L., Siddique, T. (1994) Science 264, 1772-1775
23. Adachi, H., Kume, A., Li, M., Nakagomi, Y., Niwa, H., Do, J., Sang, C., Kobayashi, Y., Doyu, M., Sobue, G. (2001) Hum. Mol. Genet. 10, 1039-1048
24. Trojanowski, J. Q., Lee, V. M. (2003) Ann. N. Y. Acad. Sci. 991, 107-110
25. Miller, D. W., Hague, S. M., Clarimon, J., Baptista, M., Gwinn-Hardy, K., cookson, M. R., Singleton, A. B. (2004) Neurology 62, 1835-1838
26. Liu, C. W., Corboy, M. J., DeMartino, G N., and Thomas, P. J. (2003) Science 299, 408-411
27. Selkoe, D. J. (1991) Neuron 6, 487-498
28. Keller, J. N., Hanni, K. B., Markesbery, W. R. (2000) J. Neurochem. 75, 436-439
29. Hashimoto, M., Hsu, L. J., Sisk, A., Xia, Y., Takeda, A., Sundsmo, M., Masliah, E. (1998) Brain Res. 799, 301-306
30. Khlistunova, I., Biernat, J., Wang, Y., Pickhardt, M., von Bergen, M., Gazova, Z., Mandelkow, E., Mandelkow, E. M. (2005) J. Biol. Chem. 24, in press
31. Benaroudj, N., Goldberg, A. L. (2000) Nat. Cell. Biol. 2, 833-839
32. Groll, M., Bajorek, M., Kohler, A., Moroder, L., Rubin, D. M., Huber, R., Glickman, M. H., Finley, D. (2000) Nature Struct. Biol. 7, 1062-1067
33. Lowe, J., Stock, D., Jap, B., Zwickl, P., Baumeister, W., Huber, R. (1995) Science 268, 533-539
34. Patel, Y. J., Payne Smith, M. D., de Belleroche, J., Latchman, D. S. (2005) Brain Res. Mol. Brain Res. 134, 256-274

### INDUSTRIAL APPLICABILITY

Use of the expression construct of the present invention allows inhibition of the formation of aggregates by an aggregative protein in eukaryotic cells. Therefore, the expression construct of the present invention can be used for treating, preventing and studying (for example, studying for the purpose of investigating causes of diseases or establishing the treatment method thereof) diseases in which the aggregative protein is involved in the onset or the development of the diseases.

The present invention is not limited to the description of the above exemplary embodiments and Examples. A variety of modifications, which are within the scopes of the following claims and which are easily achieved by a person skilled in the art, are included in the present invention.

Contents of the theses, Publication of Patent Applications, Patent Publications, and other published documents referred to in this specification are herein incorporated by reference in its entity.

[Sequence List]

1. An expression construct for degrading an aggregative protein containing a nucleic acid sequence encoding a proteasome of archaea and being operatively connected to a promoter for a eukaryotic cell.

2. The expression construct according to claim 1, wherein the nucleic acid sequence encodes archaeal proteasome α subunit and/or β subunit.

3. The expression construct according to claim 2, wherein the a subunit includes an amino acid sequence set forth in SEQ ID NO: 1 or an amino acid sequence that is different from the amino acid sequence set forth in SEQ ID NO: 1 only in a part that does not substantially affect a function of the proteasome α subunit; and
the β subunit includes an amino acid sequence set forth in SEQ ID NO: 3 or an amino acid sequence that is different from the amino acid sequence set forth in SEQ ID NO: 3 only in a part that does not substantially affect a function of the proteasome β subunit.

4. The expression construct according to claim 1, wherein the nucleic acid sequence includes a DNA sequence set forth in SEQ ID NO: 2 and/or a DNA sequence set forth in SEQ ID NO: 4.

5. The expression construct according to claim 1, wherein the archaea belongs to the Methanosarcina genus.

6. The expression construct according to claim 1, wherein the archaea is Methanosarcina mazei.

7. The expression construct according to claim 1, wherein the promoter for a eukaryotic cell is a promoter for a mammalian.

8. The expression construct according to claim 1, wherein the aggregative protein is a protein selected from the group consisting of mutant superoxide dismutase 1, an androgen receptor having an abnormally extended polyglutamine chain, α-synuclein, and tau.

9. A method of inhibiting an aggregative protein from forming an aggregate in a target eukaryotic cell, the method comprising introducing the expression construct according to claim 1 into the target eukaryotic cell.

10. A method of inhibiting an aggregative protein from forming an aggregate in a target eukaryotic cell, the method comprising forcedly expressing archaeal proteasome α subunit and β subunit in the target eukaryotic cell.

11. The method according to claim 10, wherein the archaea belongs to the Methanosarcina genus.

12. The method according to claim 10, wherein the archaea is Methanosarcina mazei.

13. The method according to claim 9, wherein the target eukaryotic cell is a human cell.

14. The method described in claim 9, wherein the target eukaryotic cell is an isolated human cell.

15. The method described in claim 9, wherein the target eukaryotic cell is a non-human mammalian cell.

16. A use of a proteasome of archaea to prepare an expression construct for degrading an aggregative protein or to inhibit an aggregative protein from forming an aggregate in a target eukaryotic cell.

17. The expression construct according to claim 2, wherein the archaea belongs to the Methanosarcina genus.

18. The expression construct according to claim 2, wherein the archaea is Methanosarcina mazei.

19. The method according to claim 10, wherein the target eukaryotic cell is a human cell.

20. The method described in claim 10, wherein the target eukaryotic cell is an isolated human cell.


SEQUENCE LISTING (9 sequences)

SEQ ID NO: 1 - protein, 249 aa, Methanosarcina mazeii
MQMAPQMGYDRAITVFSPDGRLFQVEYAREAVKRGTTAVGIKAADGVVLLVDKRITSRLVEAESIEKIFQIDDHIGAATSGLVADARSLVDRARVEAQVNRVSYDELIGVEVISKKICDHKQTYTQYGGVRPYGTALLIAGVDDNKPRLFETDPSGALLEYKATAIGAGRNAVVEVFEADYREDMNIDAAILLGMDALYKAAEGKFDAGTLEVGVVSLEDKKFRKLGPEEVENYVHQILEKHKGNENKE

SEQ ID NO: 2 - DNA, 750 bp, Methanosarcina mazeii
atgcagatggcaccacagatgggttatgacagggcaattacggttttcagccctgacggaagacttttccaggtagaatatgcccgcgaagcggtcaaaaggggaacaacagccgtaggaatcaaggcagccgatggggtagtgctgctggttgacaagcgaataacaagcaggcttgtggaagccgaatcaatcgaaaaaatattccagattgacgaccacataggagccgcaacctcagggcttgtggcagatgcccgctctcttgttgacagggcccgtgtagaagcccaggtaaacagggtttcatatgacgaactcattggtgtggaggttatctccaagaaaatctgtgaccacaagcagacatacacccagtacggaggggttcgcccttacggaaccgcactcctgattgcaggcgtggacgacaacaagccaaggctttttgagactgacccgagcggcgctctccttgagtacaaggcaacagccataggcgcaggcagaaatgcagtcgtcgaggtctttgaggcagactacagggaagatatgaacattgatgctgctatccttctcgggatggatgctctttataaagctgctgaaggcaagttcgatgccggaactcttgaagtgggtgttgtgtcccttgaggataagaaattcaggaaattaggtcctgaagaagttgagaactatgttcatcagatccttgagaaacacaaaggaaatgagaacaaagaataa

SEQ ID NO: 3 - protein, 210 aa, Methanosarcina mazeii
MDNDKYLKGTTTVGVVCTDGIVLASEQRATMGHFIASKTAKKVYQIDDLVGMTTAGSVGDAQQLVRLVSVESQLYKMRRNESMTIKGIATLMSNFLNANRYYPMMVQLLIGGVDKNGPAIYSLDPMGGSIEETRISATGSGSPMAYGVLEDQYREDIAVKEGLDLAIRAIHNATKRDSASGENIDVVVITKEAFKRLDPEEVKSRRALLN

SEQ ID NO: 4 - DNA, 633 bp, Methanosarcina mazeii
atggataatgacaaatatttaaagggcacaactaccgtaggagtagtttgtaccgacggaattgtgctcgcaagtgaacagcgagccactatggggcatttcatcgcaagcaaaactgcaaagaaagtttaccagatagatgacctggtagggatgaccactgccggttcggtaggggacgctcagcagctcgtgcgattggtaagtgtggaatcacagctctacaagatgcgcaggaacgaatccatgacgatcaaagggattgccactttgatgtccaatttcttgaatgctaaccgctattatcctatgatggttcagctccttatcggaggggttgacaaaaacggtcctgcaatctactcactggaccctatgggaggaagcattgaagagacaagaatctcagcaaccggctcgggttctcccatggcctacggagtgctggaagaccagtacagagaggatatagctgtaaaagaaggtcttgatcttgcgattcgagcaattcacaatgcaacgaaaagagactcagcttccggagaaaatatcgatgtagtcgtaattacaaaggaggcattcaaaaggttggatcctgaagaagtaaaatccagaagagctttattaaactaa

SEQ ID NO: 5 - DNA, 35 bp, Artificial
  PCR primer
gcgggtaccccaccatgcagatggcaccacagatg

SEQ ID NO: 6 - DNA, 36 bp, Artificial
  PCR primer
cgcctcgagttattctttgttctcatttcctttgtg

SEQ ID NO: 7 - DNA, 37 bp, Artificial
  PCR primer
gcgggtaccccaccatgacggttttcagccctgacgg

SEQ ID NO: 8 - DNA, 38 bp, Artificial
  PCR primer
gcctctagaccaccatggataatgacaaatatttaaag

SEQ ID NO: 9 - DNA, 28 bp, Artificial
  PCR primer
gcgaccggtgtttcctaaagctcttctg